(19) United States
(12) Patent Application Publication
(10) Pub. No.: US 2007/0281881 A1
(43) Pub. Date: Dec. 6, 2007

(54) Methods And Compositions Comprising Non-Peptide Small Molecules That Solubilize Beta Amyloid Peptide Fiber

(76) Inventors: Yves Claude Nicolau, Newton, MA (US); Alexey Eliseev, Brookline, MA (US); Jean-Marie Lehn, Strasbourg (FR)

Correspondence Address: JOHN S. PRATT, ESQ;KILPATRICK STOCKTON, LLP, 1100 PEACHTREE STREET, ATLANTA, GA, 30309, US

(21) Appl. No.: 10/590,071

(22) PCT Filed: Feb. 18, 2005
(86) PCT No.: PCT/US05/05381

§ 371 Date: Jul. 17, 2007

(30) Foreign Application Priority Data
Feb. 20, 2004 (US) 10/783699

(51) Int. Cl. A61K 38/17; C40B 30/06; C40B 40/10

(52) U.S. Cl. 514/2; 506/9; 506/18

(57) ABSTRACT

The present invention comprises novel compositions and methods for the treatment of neurological diseases associated with beta amyloid peptide. In particular, the present invention is directed to Aβ fiber solubilization, for the treatment of amyloid-associated diseases such as Alzheimer's disease, and is described herein as being achieved by using small molecules binding to Aβ fiber The present invention further comprises novel methods of designing and screening compositions for the treatment of neurological disease wherein the design aspect comprises the modeling of molecular scaffolds having optimal structural characteristics that enable the development of small molecule compositions of non-peptide nature having desirable solubilization effects on amyloid plaques. In one embodiment, the design of the compounds comprises a molecular structure with three chemical domains that include a melatonin-like domain, a nicotine-like domain and a peptide-like domain for binding to Aβ.

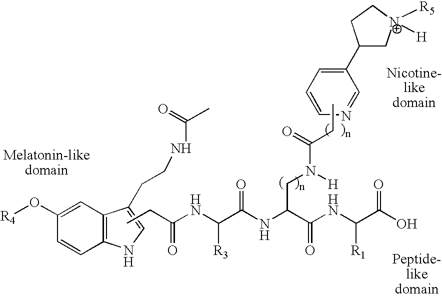

### FIELD OF THE INVENTION

The present invention is related to methods for designing and screening for novel compositions useful for treating neurological diseases associated with amyloid beta plaque formation such as Alzheimer's disease, hemorragic stroke due to cerebral amyloid angiopathy, amyloidosis and epilepsy. In particular, the present invention comprises methods for designing and screening for novel compositions for destroying plaques formed by aggregation of fibers of amyloid peptide in its beta sheet conformation (Aβ).

### BACKGROUND OF THE INVENTION

Neurological diseases are becoming an increased focus as the world's population ages and scientists' knowledge of the brain increases. Modern medicine is extending the lifespan of the human body, however the treatment of neurological disease has not advanced proportionately and mental faculties still degenerate. Neurological disease associated with amyloid plaques is of particular importance. The most well-known neurological disease associated with amyloid plaques is Alzheimer's disease (AD) although other neurodegenerative diseases are associated with amyloid plaques. Such diseases include hemorragic stroke due to cerebral amyloid angiopathy, amyloid angiopathy amyloidosis and epilepsy.

Alzheimer's disease is a progressive disease of the brain that is characterized by impairment of memory and a disturbance in at least one other thinking function (for example, language or perception of reality). Alzheimer's disease is not a normal part of aging and is not an inevitable occurrence. Rather, it is one of the ‘dementing disorders’ which are a group of brain diseases that result in the loss of mental and physical functions.

The main risk factor for AD is increased age. The prevalence of AD increases with age, with 10% of people over age 65 and 50% of those over 85 developing AD. The number of individuals with AD is expected to be 14 million by the year 2050. In 1998, the annual cost for the care of patients with AD in the United States was approximately $40,000 per patient.

There are also genetic risk factors for AD. The presence of several family members with AD has suggested that, in some cases, heredity may influence the development of AD. A genetic basis has been identified through the discovery of mutations in several genes that cause AD in a small subgroup of families in which the disease has frequently occurred at relatively early ages (beginning before age 50). Some evidence points to chromosome 19 as implicated in certain other families in which the disease has frequently developed at later ages.

With the exception of rare cases of familial AD, in which the disease is caused by mutations (changes in the DNA) of a single gene, most cases of AD are probably caused by a variety of cooperating factors. Cases without a family history are called “sporadic.” The study of familial AD, however, has uncovered several proteins that are not only important for familial, but also for sporadic AD. These proteins include amyloid precursor protein (APP) and two presenilins (presenilin I and presenilin II). APP is a major component of amyloid plaques which are caused by abnormal deposit of proteins in the brain.

The degradation of APPs likely increases their propensity to aggregate in plaques. Presenilins, on the other hand, are involved in the cleavage of APP. Mutations in the genes that encode APPs and the presenilins can cause AD. This means that individuals carrying these mutations have a very high probability of developing AD.

The symptoms of AD manifest slowly and the first symptom may only be mild forgetfulness. In this stage, individuals may forget recent events, activities, the names of familiar people or things and may not be able to solve simple math problems. As the disease progresses, symptoms are more easily noticed and become serious enough to cause people with AD or their family members to seek medical help. Mid-stage symptoms of AD include forgetting how to do simple tasks such as grooming, and problems develop with speaking, understanding, reading, or writing. Later stage AD patients may become anxious or aggressive, may wander away from home and ultimately need total care.

Scientific evidence demonstrates that AD results from an increase in the production or accumulation of beta-amyloid protein in plaques that leads to nerve cell death. Loss of nerve cells in strategic brain areas, in turn, causes reduction in the neurotransmitters and impairment of memory.

Presently, the only definite way to diagnose AD is to identify plaques and tangles in brain tissue in an autopsy after death of the individual. Therefore, doctors can only make a diagnosis of “possible” or “probable” AD while the person is still alive. Using current methods, physicians can diagnose AD correctly up to 90 percent of the time using several tools to diagnose “probable” AD. Physicians ask questions about the person's general health, past medical problems, and the history of any difficulties the person has carrying out daily activities. Behavioral tests of memory, problem solving, attention, counting, and language provide information on cognitive degeneration and medical tests-such as tests of blood, urine, or spinal fluid, and brain scans can provide some further information.

The management of AD consists of medication-based and non-medication based treatments. Treatments aimed at changing the underlying course of the disease (delaying or reversing the progression) have so far been largely unsuccessful. Medicines that restore the deficit (defect), or malfunctioning, in the chemical messengers of the nerve cells (neurotransmitters), such as the cholinesterase inhibitors (ChEIs), have been shown to improve symptoms. Medications are also available to address the psychiatric manifestations of AD.

Cholinesterase inhibitors, such as tacrine and rivastgmine, are currently the only class of agents that are approved by the FDA for the treatment of AD. These agents are medicines that restore the defect, or malfunctioning, in the chemical neurotransmission in the brain. ChEIs impede the enzymatic degradation of neurotransmitters thereby increasing the amount of chemical messengers available to transmit the nerve signals in the brain.

For some people in the early and middle stages of the disease, the drugs tacrine (COGNEX®, Morris Plains, N.J.), donepezil (ARICEPT®, Tokyo, JP), rivastigmine (EXELON®, East Hanover, N.J.), or galantamine (REMINYL®, New Brunswick, N.J.) may help prevent some symptoms from becoming worse for a limited time. Another drug, memantine (NAMENDA®, New York, N.Y.), has been approved for treatment of moderate to severe AD. Also, some medicines may help control behavioral symptoms of AD such as sleeplessness, agitation, wandering, anxiety, and depression. Treating these symptoms often makes patients more comfortable and makes their care easier for caregivers. Unfortunately, despite significant treatment advances showing that this class of agents is consistently better than a placebo, the disease continues to progress despite treatment, and the average effect on mental functioning has only been modest. ChEIs also have side effects that include gastrointestinal dysfunction, liver toxicity and weight loss.

Advances in the understanding of the brain abnormalities that occur in AD are hoped to provide the framework for new targets of treatment that are more focused on altering the course and development of the disease. Many compounds, including anti-inflammatory agents, are being actively investigated. Clinical trials using specific cyclooxygenase inhibitors (COX-2), such as rofecoxib and celecoxib, are also underway.

Another factor to consider when developing new drugs is the ease of use for the target patients. Oral drug delivery—specifically tablets, capsules and softgels—account for 70% of all dosage forms consumed because of patient convenience. Drug developers agree that patients prefer oral delivery rather than subjecting themselves to injections or other, more invasive forms of medicinal administration. Small molecule drugs are preferable to antibody or peptide-based therapies because of the convenience and ease of packaging in formulations for ease of consumption. Formulations resulting in low dosing intervals (i.e. once a day or sustained release) are also preferable. The ease of administering small molecules in oral dosage forms result in an increase of patient compliance during treatment.

What is needed therefore, are effective compositions and methods for addressing the complications associated with neurological disease associated with amyloid plaque formation such as Alzheimer's disease. In particular what is need are novel small molecule pharmaceuticals capable of counteracting the physiological manifestations of the disease such as the formation plaques associated with aggregation of fibers of the amyloid peptide in its beta sheet conformation (Aβ). Such compositions would preferably encourage patient compliance.

### SUMMARY OF THE INVENTION

The present invention is directed to methods of designing and screening for novel compositions for the treatment of neurological disease associated with amyloid plaque formation. In particular, the present invention is directed to methods of designing and screening compositions for Aβ fiber solubilization, which is considered to be an approach for the treatment of Alzheimer's disease, and is described herein as being achieved by using small molecules of non-peptide nature. A novel class of compounds is described herein that may provide such a solubilization effect. In one embodiment, the design of the compounds comprises a molecular structure with three chemical domains that include a melatonin-like domain, a nicotine-like domain and a peptide-like domain for binding to Aβ. A single compound comprising these three chemical domains provides a preferable structure for binding amyloid peptide for solubilization.

Two mainstream strategies in the research of active molecules are molecular design and high throughput screening of chemical libraries built on previous pharmacophore knowledge. The methods described herein focus on the strategic and scientific aspects of thermodynamic stabilization of soluble forms of Aβ. The drug design methods include designing compounds and generating a combinatorial library of candidate compounds predicted to bind Aβ. Methods of screening compounds for activity on solubilization of Aβ fibers in vitro and in vivo are also described herein.

Accordingly, an object of the present invention is to provide methods of drug design and novel compositions for the treatment of neurological diseases.

It is another object of the present invention is to provide methods of drug design and novel compositions for the treatment of diseases associated with amyloid plaque formation.

It is another object of the present invention is to provide methods of drug design and novel compositions for amyloid fiber solubilization.

Yet another object of the present invention is to provide methods of drug design and novel compositions for amyloid fiber solubilization comprising small non-peptide molecules that bind to and solubilize amyloid protein.

Another object of the present invention is to provide methods of drug design and novel compositions for the treatment of Alzheimer's disease.

An additional object of the present invention is to provide methods of drug design and novel compositions for the treatment of Alzheimer's disease comprising small non-peptide molecules that solubilize Alzheimer's Aβ peptide fiber.

Another object of the present invention is to provide novel small non-peptide compositions comprising specific structural domains of chemical entities.

Another object of the present invention is to provide novel small non-peptide compositions comprising a melatonin-like structural domain, a nicotine-like structural domain or a peptide-like structural domain for solubilizing Aβ peptide fiber.

Yet another object of the present invention is to provide novel small non-peptide compositions comprising a melatonin-like structural domain, a nicotine-like structural domain and a peptide-like structural domain for solubilizing Aβ peptide fiber.

Another object of the present invention is to provide methods and compositions for reducing and preventing the formation plaques associated with aggregation of fibers of the amyloid peptide in its beta sheet conformation (Aβ).

Yet another object of the present invention is to provide novel compositions that solubilize Aβ peptide fiber that may be administered intramuscularly, intravenously, transdermally, orally, or subcutaneously.

These and other objects, features and advantages of the present invention will become apparent after a review of the following detailed description of the disclosed embodiment and the appended claims.

### BRIEF DESCRIPTION OF THE FIGURES

FIG. 1 provides a schematic showing a chemical approach to combinatorial discovery of β-sheet breaking small molecules.

FIG. 2 provides a schematic showing the “morphomer” concept.

FIG. 3 provides a schematic showing molecular adaptation of morphomers to the target leading to the formation of strong complexes.

FIG. 4 provides a general strategy of small molecules-β-sheet breakers for therapy of Alzheimer's disease.

FIG. 5 provides a schematic for screening of small molecules for Aβ solubilization activity.

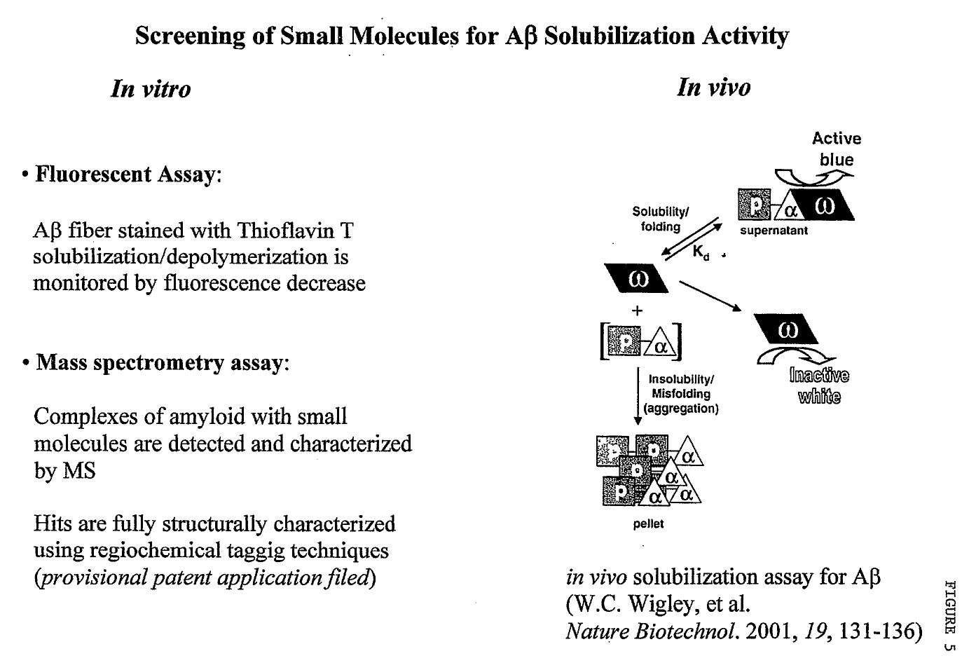

FIG. 6 provides a schematic demonstrating the conformational changes of Aβ in micelles and in solutions of trifluoroethanol/water 40:60 and water.

FIG. 7 provides a schematic showing targets on Aβ for molecular design.

FIG. 8 provides a schematic diagram showing a structure of potential small molecule compounds predicted to bind to Aβ (8A) and the manner in which the compounds are expected to bind to the helix-loop-helix conformation of Aβ (8B).

FIG. 9 provides a schematic diagram showing the combination of the melatonin-like domain, the nicotine-like domain and the peptide domain for synthesis of small molecule compounds that bind to Aβ.

### DETAILED DESCRIPTION

The present invention may be understood more readily by reference to the following detailed description of specific embodiments included herein. Although the present invention has been described with reference to specific details of certain embodiments thereof, it is not intended that such details should be regarded as limitations upon the scope of the invention. The text of the references mentioned herein are hereby incorporated by reference in their entirety, including U.S. Provisional Application Ser. No. 60/489,732 and U.S. patent application Ser. No. 10/783,699.

I. Compositions

A. Active Ingredients

The development of the Alzheimer's disease is thought to be directly related to the formation of plaques formed via aggregation of fibers of the amyloid peptide in its beta-sheet conformation (Aβ). The present invention comprises any agent capable of directly or indirectly destroying such plaques, either via solubilization of amyloid protein or by its chemical decomposition. As such, the agents described herein are useful for therapeutic intervention in neurological diseases and disorders associated with amyloid plaques and especially for Alzheimer's disease therapy.

In one embodiment, preferred compounds of the present invention include non-peptidic small molecule compounds possessing conformational diversity and the ability to bind soluble forms of amyloid protein.

The β-sheet fiber form of Aβ is formed through a slow conformational change from its soluble form, which is a combination of the a-helical and random coil structures, followed by aggregation. (Jarvet, J. et al., Am. Chem. Soc. 2000, 122:4261-4268.) Thus, one possible mechanism for solubilization of plaques is via thermodynamic stabilization of the soluble form and thereby reversing the fiber precipitation equilibrium. This concept, referred to as β-sheet breaking, has recently proven successful with a number of β-sheet breaking peptides. (Poduslo, J. F. et al. J. Neurobiol 1999, 39:371-382; Sigurdsson, E. M. et al., Neuropathol. Exp. Neurol 2000, 59:11-17).

Aβ is not a stable conformation of a large protein in water solutions, but an ill-defined ensemble of relatively low molecular mass foldamers. The low-molecular-weight peptides, primarily fragments or analogs of the native Aβ, were shown to solubilize the Aβ fiber, supposedly by forming a non-covalent complex with the soluble conformation of Aβ. Such an activity of the β-sheet breaking peptides resulted not only in the in vitro solubilization of the fiber, but also in the in vivo reduction of plaques in mice. Because Aβ is not a stable conformation of a large protein in water solutions, there has been no well-defined, validated binding pocket identified up to this point. The molecular surfaces are a concern in molecular recognition by a candidate compound. A candidate compound may be defined as any chemical compound that can bind to amyloid peptide and preferably solubilize amyloid peptide. Preferably, the candidate compounds are small (approximately molecular weight 400), lipophilic, and able to cross the blood-brain barrier while also being water soluble for diffusion through the tissues. Since concentration of Aβ in blood plasma is about 3.3 nM, in order to bind 80% of Aβ by a synthetic compound present at 10 μM, a binding constant in the range of approximately 2 μM is needed; corresponding to a binding energy of about 8 kcal/mol. The best, non-peptidic compounds that bind Aβ to date have binding constants in the 10 μM range.

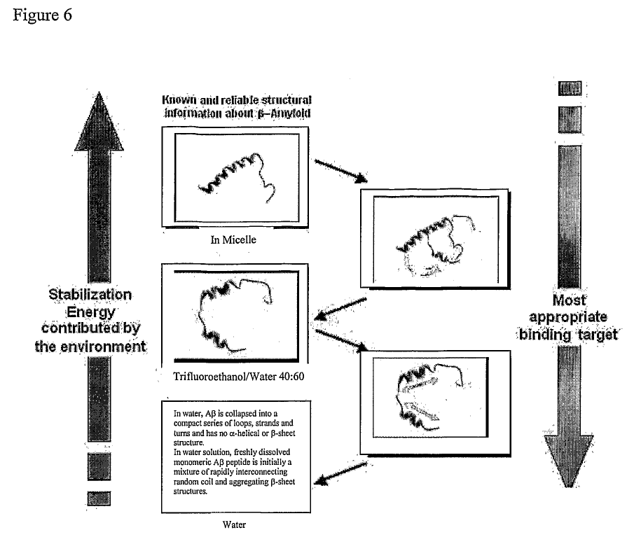

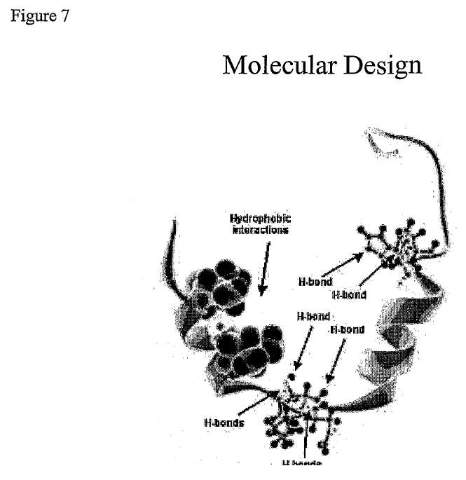

In order to design compounds to target Aβ for solubilization, an understanding of the chemistry behind the thermodynamic stabilization of soluble amyloid protein is required. One goal for drug design is to identify an actual β-amyloid fold for binding by the compound in order to achieve the thermodynamic stabilization of a soluble form. In a micelle, the amyloid peptide is in an alpha-helix conformation, with a random-coil structure at the N-terminus (FIG. 6). Part of the peptide's sequence is incorporated into the micelle's bilayer. In 40% trifluoroethanol/water, the NMR determined conformation is helix-loop-helix like, with a random-coil structure at the N-terminus. None of these conformations are predominantly present in water, where freshly dissolved amyloid peptide is initially a mixture of rapidly interconverting α-helices, random coil and aggregating β-sheet structures. The conformation of the peptide is strongly dependent on the “hydrophobicity” of the environment. Therefore, in the absence of precise structural information in water, one should consider binding the conformation of amyloid protein that is energetically the most accessible of the conformations that are possible in water. Indeed, binding this conformation is likely to provide two principal benefits: firstly, it is likely that this will provide the most stabilization energy as the initial mixture will evolve to the compound-bound single conformation; and secondly, this will maintain the water-soluble character of the bound form. The amyloid peptide form that is preferably targeted for drug design is the helix-loop-helix structure shown in FIG. 7.

The structure shown in FIG. 7 is an ideal binding target for drug design. The energies that are involved in the different types of interactions within peptidic structures, are such that in order to obtain a binding energy of preferably between 1 and 20 kcal/mol, more preferably between 5 and 15 kcal/mol, more preferably 7-12 kcal/mol and most preferably about 8 kcal/mol with an approximately 400 molecular weight compound, at least one buried (water shielded) hydrogen bond is necessary. It is difficult to achieve 8 kcal/mol binding if one is attempting to bind the alpha helix conformation described in FIG. 6, on which all possible binding sites are water-exposed. However, the helix-loop-helix conformation possesses a U-shaped binding space, in which a compound can be partially shielded from the water environment, therefore providing, a much better locus for binding than the α-helix conformation.

Some studies have reported reductions in Aβ aggregation with chemical compounds. It has been shown that nicotine binds and slows down Aβ aggregation. (Moore et al., Biochemistry. Jan. 27, 2004;43(3):819-26; Nordberg, et al., J Neurochem. May 2002;81(3):655-8; Salomon et al., Biochemistry. Oct. 22, 1996; 35(42):13568-78). Melatonin also binds and slows down Aβ aggregation (Chyan et al., J Biol Chem, 274(31):21937-21942, 1999; Papolla et al., J Biol Chem, 273(13): 7185-7188, 1998) and easily crosses the blood brain barrier. These studies report stabilizing Aβ protein and slowing down the aggregation of Aβ into plaques but they do not demonstrate solubilizing of existing plaques as a treatment for amyloid-associated disease. In addition, in contrast to the present invention, these studies are limited to individual chemical compounds present in intact form. The unique compositions of the present invention combine important and relevant domains of such chemical compounds and arrange them in specific structural conformations. The compounds of the present invention further incorporate characteristics pertinent to specific site binding on Aβ. Proline positional scanning on the Aβ clearly demonstrate the importance of residues 22, 23, 29 and 30 (respectively Glu, Asp, Gly, Ala) in the misfolding process and peptide folding studies have clearly established that the sequence 16-20 (KLVFF) (SEQ ID NO:1) of Aβ undergoes significant modification in the transition of soluble Aβ to fibrils. These design elements are summarized in FIG. 7. It would therefore be desirable to have a variety of small molecules designed according to the parameters described above incorporating elements such as optimal structural positioning and site specific binding.

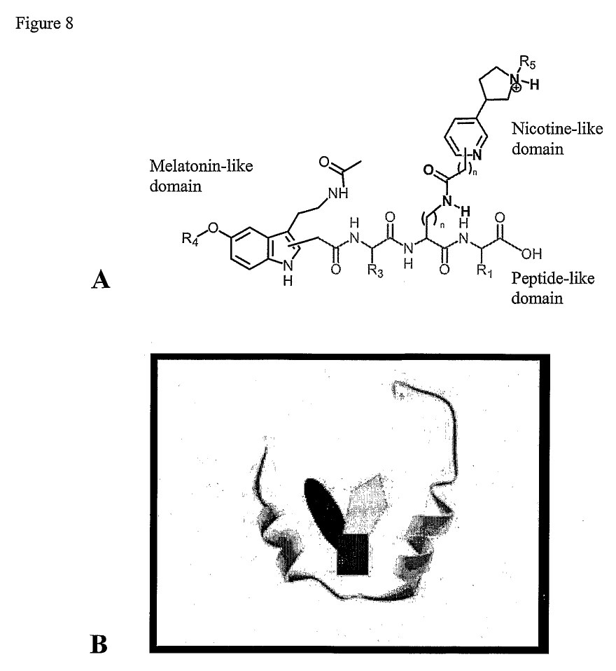

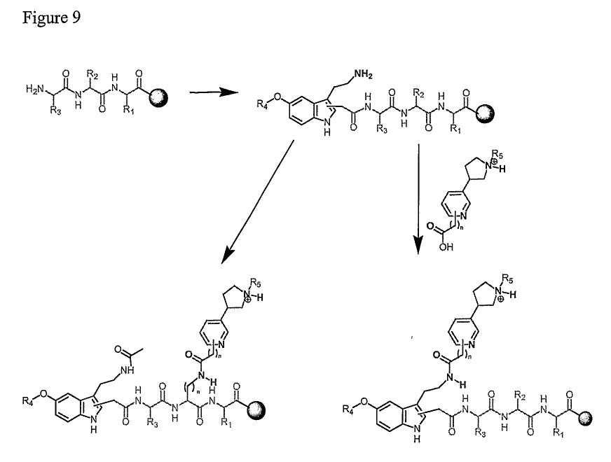

A novel feature of this invention is the design of a unique class of molecules comprising two or more molecular domains selected for their ability to target and solubilize Aβ. Specific molecules within this class may be designed with variations in the binding affinity and rate of solubilization by varying the identity of the R-group of the base chemical structure. A preferred candidate compound ideally possesses a chemical scaffold comprising multiple building blocks or chemical domains that provide it with properties for promoting binding and solubilization of Aβ fibers. In certain preferred embodiments, the preferred candidate compound ideally possesses a chemical scaffold comprising three building blocks or chemical domains. This first molecular domain comprises a melatonin-like domain, for binding to the hydrophobic inner face of the Aβ peptide. The second molecular domain comprises a nicotine-like domain, for binding the histidine cluster (His 13 and His 14) near the N-terminus of the Aβ peptide. The third molecular domain comprises a peptide-like unit, suitable for H-bonds in the water-hidden loop-like area. Compounds with these three domains are schematically shown binding to Aβ in FIG. 8. Combinatorial libraries of compounds with these three domains are screened for small molecules with optimal activity on solubilizing Aβ fibers. Although compounds containing all three domains represent one preferred embodiment, compounds possessing only two of these domains are also useful for binding Aβ. These compounds may contain a nicotine-like domain and a melatonin-like domain, a nicotine-like domain and a melatonin-like domain or a melatonin-like domain and a peptide-like domain. As demonstrated in FIG. 9, the melatonin-like domain may be chemically bonded to either the nicotine-like domain or the peptide-like domain and the melatonin-like and peptide-like domains may also be bonded together.

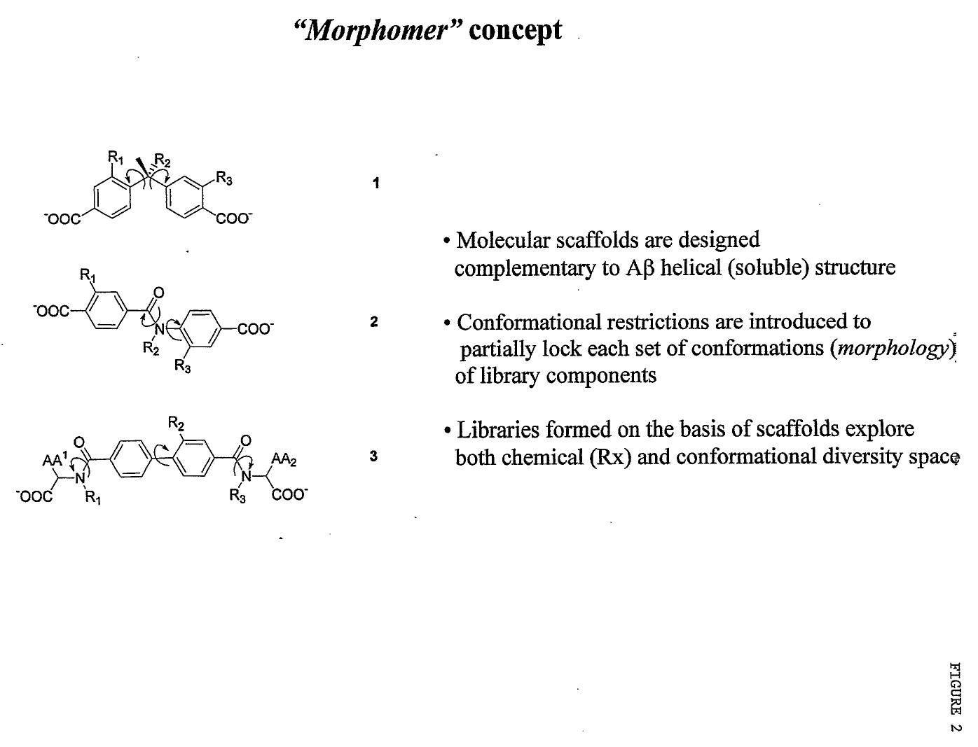

The methods of designing and screening candidate compounds utilize a new concept in the design of the entities engaged in the molecular recognition, i.e. in formation of non-covalent complexes with biological targets. (Bunyapaiboonsri, T. et al., 2003, J Med Chem. 46(26):5803-11; Ramström O. and Lehn J-M., 2002, Nature Reviews, 1:26-36). Biological targets generally possess binding sites for candidate molecules in proteins and other cellular molecules. The small molecule solubilization compositions of the present invention are non-covalent complexes of small molecules with particular sequences of target proteins. They occur in different conformational states and permit the design of drugs capable of targeting and modifying the conformational state of targeted proteins thereby rendering them harmless. The custom designed small molecules of the present invention (also known as Morphomers™) include a number of bonds, around which the intramolecular rotation is slower than around average single bonds, such as e.g. sp³ carbon-carbon bond. Although the conformational change occurs in solution, it is sufficiently slow so that corresponding conformations can be observed, for example, as individual peaks in the NMR spectrum of the compounds. The examples of such conformationally restricted bonds are shown with arrows in structures 1-3. (FIG. 2).

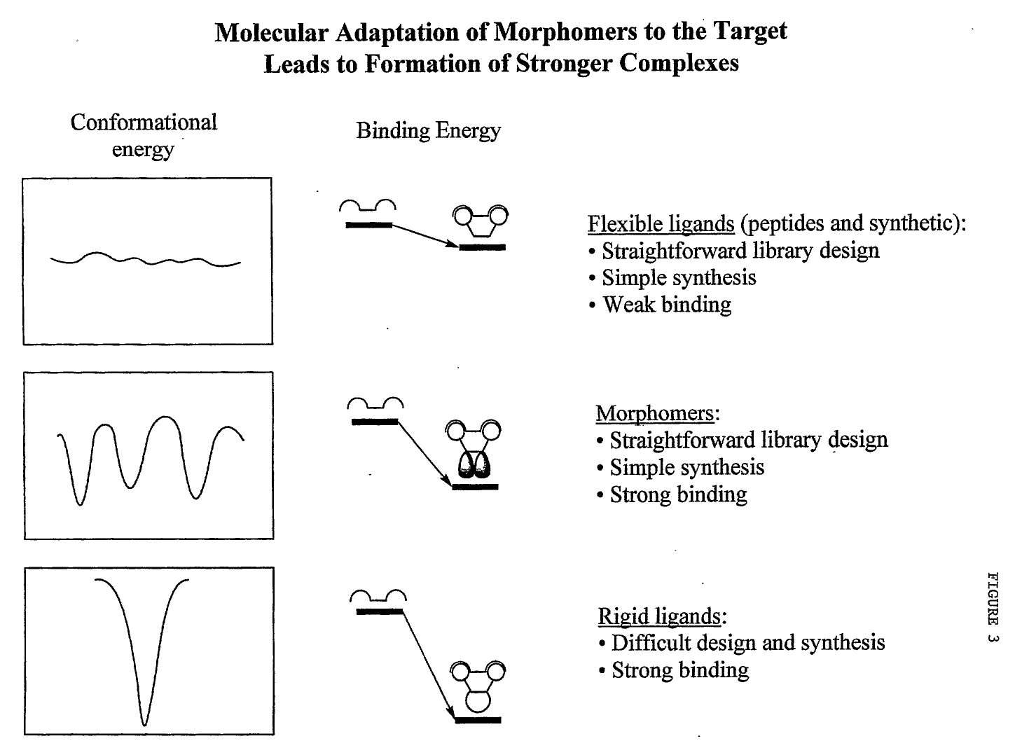

Small molecule design according to the novel methods described herein enables the identification of novel classes of compounds that “break” the pathological β-sheet conformation of proteins (such as amyloid beta protein) by shifting the equilibrium to the soluble form (α-helix) of the protein target. The different conformational states of small molecules, therefore form a sub-library of the main combinatorial library. Two properties of the small molecules resulting from the novel design methods described herein are important for pharmaceutical applications: (i) binding energy to the target differs between small molecules; (ii) binding energy of small molecules that have a better geometrical and functional fit to the target is higher than that of a similar compound with unrestricted conformations, as shown in FIG. 3.

B. Salts and Derivatives

Although described above with reference specific to compounds, one can also utilize enantiomers, stereoisomers, metabolites, derivates and salts of the active compounds. Methods for synthesis of these compounds are known to those skilled in the art. The term “pharmaceutically acceptable salt” means those salts which retain the biological effectiveness and properties of the compounds used in the present invention, and which are not biologically or otherwise undesirable. Such salts may be prepared from inorganic and organic bases. Salts derived from inorganic bases include, but are not limited to, the sodium, potassium, lithium, ammonium, calcium, and magnesium salts. Salts derived from organic bases include, but are not limited to, salts of primary, secondary and tertiary amines, substituted amines including naturally-occurring substituted amines, and cyclic amines, including isopropylamine, trimethylamine, diethylamine, triethylamine, tripropylamine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, and N-ethylpiperidine. It should also be understood that other carboxylic acid derivatives, for example carboxylic acid amides, including carboxamides, lower alkyl carboxamides, di(lower alkyl)carboxamides, could be used.

Examples of pharmaceutically acceptable salts include, but are not limited to, mineral or organic acid salts of basic residues such as amines, and alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. Conventional non-toxic salts include those derived from inorganic acids such as hydrochloric, hydrobromic, sulfuric, sulfamic, phosphoric and nitric acid; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, tolunesulfonic, methanesulfonic, ethane disulfonic, oxalic and isethionic acids. The pharmaceutically acceptable salts can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed. (Mack Publishing Company, Easton, Pa., 1985, p. 1418) the disclosure of which is hereby incorporated by reference.

A prodrug is a covalently bonded substance which releases the active parent drug in vivo. Prodrugs are prepared by modifying functional groups present in the compound in such a way that the modifications are cleaved, either in routine manipulation or in vivo, to yield the parent compound. Prodrugs include compounds wherein the hydroxy or amino group is bonded to any group that, when the prodrug is administered to a mammalian subject, cleaves to form a free hydroxyl or free amino, respectively. Examples of prodrugs include, but are not limited to, acetate, formate and benzoate derivatives of alcohol and amine functional groups.

A metabolite of the above-mentioned compounds results from biochemical processes by which living cells interact with the active parent drug or other formulas or compounds of the present invention in vivo. Metabolites include products or intermediates from any metabolic pathway.

C. Formulations and Excipients

The active compounds (or pharmaceutically acceptable salts thereof) may be administered per se or in the form of a pharmaceutical composition wherein the active compound(s) is in admixture or mixture with one or more pharmaceutically acceptable carriers, excipients or diluents. Pharmaceutical compositions may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

The active compounds can be formulated in a pharmaceutical composition comprising a pharmaceutically acceptable carrier and an effective amount of active ingredient to solubilize amyloid beta protein tangles and plaques. For example, the pharmaceutical composition can comprise a pharmaceutically acceptable carrier and an effective amount of a small molecule such as one embodied by the formula in FIG. 8A.

The active ingredient can be administered orally in solid dosage forms, such as capsules, tablets and powders, or in liquid dosage forms, such as elixirs, syrups and suspensions. It can also be administered parenterally, in sterile liquid dosage forms. Additives may also be included in the formulation to enhance the physical appearance, improve stability, and aid in disintegration after administration. Liquid dosage forms for oral administration can contain coloring and flavoring to increase patient acceptance. Typical additives include diluters, binders, lubricants, and disintegrants. Gelatin capsules contain the active ingredient and powdered carriers, such as lactose, starch, cellulose derivatives, magnesium stearate, stearic acid, and the like. Similar diluents can be used to make compressed tablets. Both tablets and capsules can be manufactured as sustained release products to provide for continuous release of medication over a period of hours or days. Sustained release products can also be formulated for implantation or transdermal/transmucosal delivery. Such formulations typically will include a polymer that biodegrades or bioerodes thereby releasing a portion of the active ingredient. The formulations may have the form of microcapsules, liposomes, solid monolithic implants, gels, viscous fluids, discs, or adherent films.

Injectable preparations, for example, sterile injectable aqueous or oleaginous suspensions, can be formulated according to the known art using suitable dispersing or wetting agents and suspending agents. The active ingredient is preferably formulated in a pharmaceutically acceptable carrier. Such carriers are known to one of skill in the art. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid are useful in the preparation of injectables. Dimethyl acetamide, surfactants including ionic and non-ionic detergents, and polyethylene glycols can be used. Mixtures of solvents and wetting agents such as those discussed above are also useful.

Solutions for parenteral administration preferably contain a water-soluble salt of the active ingredient, suitable stabilizing agents, and if necessary, buffer substances. Typically, water, suitable oil, saline, aqueous dextrose (glucose), and related sugar solutions and glycols such as propylene glycol or polyethylene glycols are suitable carriers for parenteral solutions. Antioxidizing agents such as sodium bisulfite, sodium sulfite, or ascorbic acid, either alone or combined, are suitable stabilizing agents. Also used are citric acid and its salts, and sodium EDTA. In addition, parenteral solutions can contain preservatives, such as benzalkonium chloride, methyl- or propyl-paraben and chlorobutanol. Suitable pharmaceutical carriers are described in Remington's Pharmaceutical Sciences, supra, a standard reference text in this field.

The compounds, or pharmaceutically acceptable salts thereof, can be formulated as pharmaceutical compositions, including their polymorphic variations. Such compositions can be administered orally, buccally, parenterally, by inhalation spray, rectally, intradermally, transdermally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, or intrasternal injection, or infusion techniques. In the preferred embodiment the composition is administered orally.

Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

The amount of active ingredient that can be combined with the carrier materials to produce a single dosage form will vary depending upon the patient and the particular mode of administration.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

The candidate compound may be administered orally, bucally, parenterally, topically, rectally, vaginally, by intranasal inhalation spray, by intrapulmonary inhalation or in other ways known to one of skill in the pharmaceutical arts.

Therapeutically effective amounts for use in humans can be determined from animal models. For example, a dose for humans can be formulated to achieve circulating concentration that has been found to be effective in animals. Useful animal models for Alzheimer's disease are known in the art. (German et al. Rev Neurosci. 2004;15(5):353-69; Moolman, et al., J Neurocytol. 2004, 33(3):377-87). In particular, the following references provide a suitable primate model of AD. (Gandy et al. Alzheimer Dis Assoc Disord. January-March 2004;18(1):44-6).

Effective amounts for use in humans can be also be determined from human data for the compounds used to solubilize AB plaques. Patient doses for oral administration of the compound typically range from about 1 μg-10 gm/day. The dosage may be administered once per day or several or multiple times per day. The amount of the compound will of course, be dependent on the subject being treated, the severity of the affliction, the manner of administration and the judgment of the prescribing physician.

The compound can be administered intrathecally to ensure the entry of the compound into the cerebrospinal fluid. Intrathecal drug delivery systems are commercially available (Medtronic, Inc., Minneapolis, Minn.) and can be implanted into the patient for release of medication at constant or variable flow rates. Intrathecal drug delivery systems are composed of two implantable components: an infusion pump and an intraspinal catheter. The pump is placed abdominally in a subcutaneous pocket, while the catheter is inserted into the intrathecal space of the spine, tunneled under the skin and connected to the pump. Since larger doses of drug are needed for oral or intravenous administration to cross the blood-brain barrier, intrathecal injection of the drug significantly reduces the amount of drug needed for a physiological effect. An intrathecal administration of a drug can be reduced to approximately 1/300 of the required oral dose.

II. Drug Design and Development

A. Synthesis of Compounds

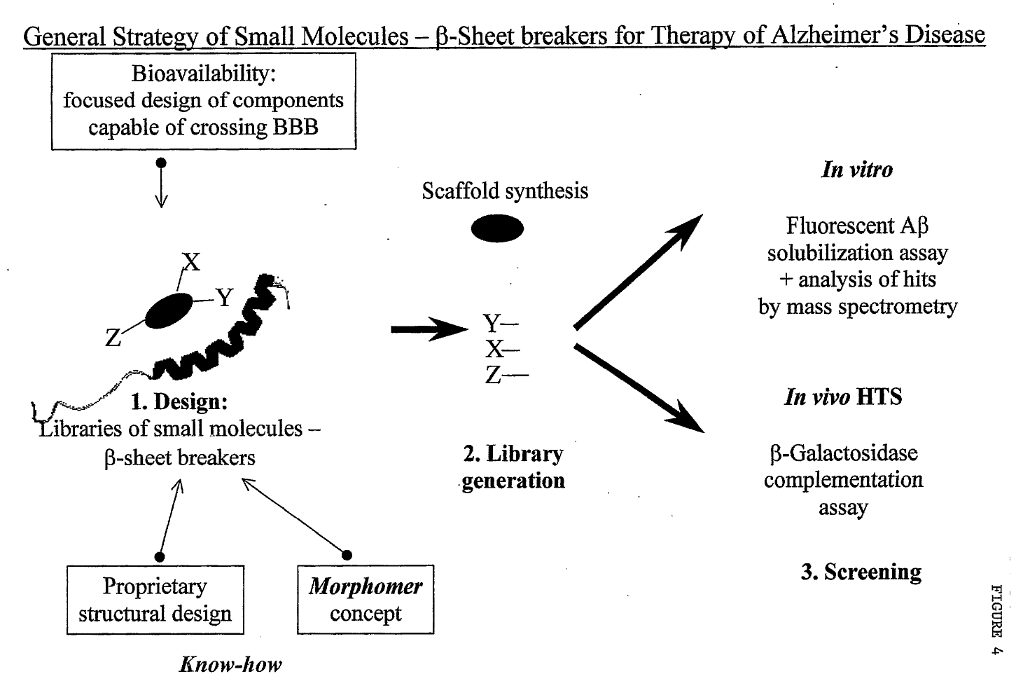

A general strategy for the discovery of candidate compounds is shown in FIG. 4. The strategy generally consists of computer-aided design of the molecular scaffolds, their synthesis, formation of the combinatorial libraries, and their screening in vitro and in vivo using known procedures.

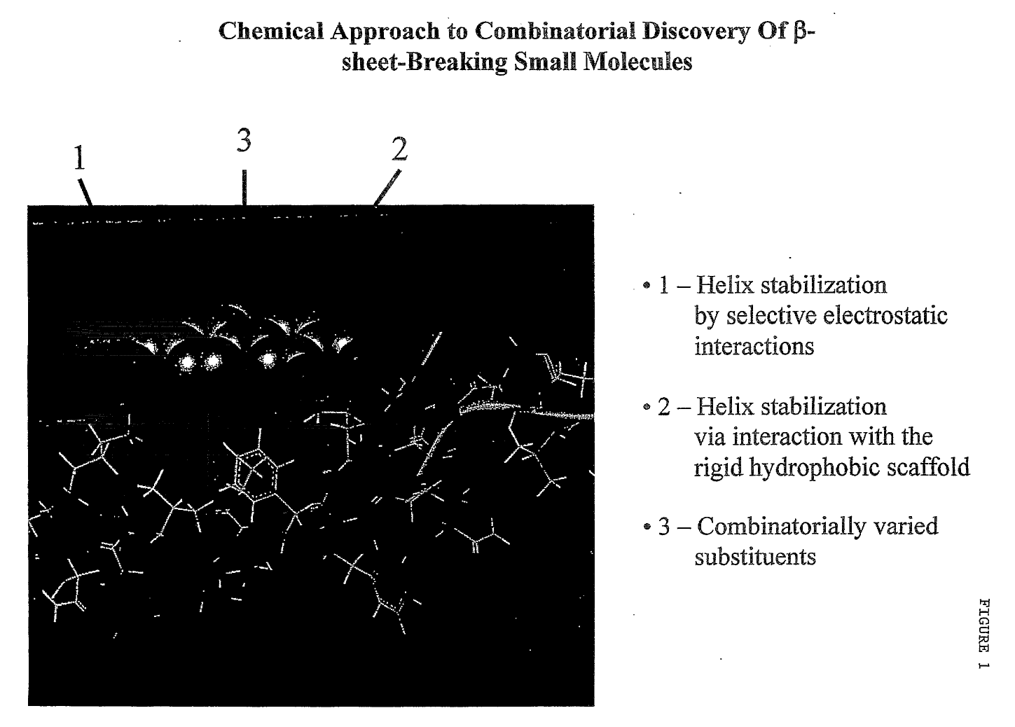

The small molecules described herein are generated from combinatorial libraries of compounds based on the scaffold structures 1-3 (FIG. 2) or similar compounds that can form non-covalent complexes with the soluble form of Aβ. As shown in FIG. 1, the binding of the compounds to Aβ is achieved via combination of electrostatic, hydrophobic, hydrogen-bonding and other kinds of non-covalent interactions with the Aβ a-helical form. The scaffold structures 1-3 have been designed with the aid of computer-assisted molecular modeling, taking into account the projected properties of the library components to facilitate their crossing the blood-brain barrier. (Begley, D. J. Journal of Pharmacy & Pharmacology 1996, 48, 136-146.; Cecchelli, R. et al., Adv. Drug Deliv. Rev 1999, 36, 165-178). The substituents R₁-R₃ and AA₁, AA₂ are varied within combinatorial libraries of the potential β-sheet breakers and the resulting component show different activity in the solubilization assays described below.

With advances in contemporary drug design, it is now possible to generate combinatorial libraries with multiple candidate molecules and submit them to high-throughput screening processes. Examples of such methods are disclosed in U.S. Pat. Nos. 6,828,435, 6,828,143, 6,828,427, 5,324,483, 5,288,514, and PCT W091/19735 and PCT WO 94/26775.

Compounds are preferably synthesized using the commercially available IRORI™ technology (Discovery Partners International, San Diego, Calif.). This technology includes semi-porous micro-reactor tubes each containing a solid phase synthesis resin and a radiofrequency (Rf) tag as a miniature memory device. IRORI's patented “direct sorting” technology uses the Rf tags to encode the micro-reactors. After each chemical synthesis step, a software program sorts the micro-reactors to the next synthesis step. A selected group of microreactors can be incubated in a reaction vessel for each synthesis step and then separated and sorted again for subsequent steps in order to add subsequent chemical groups and side chains in the desired order and orientation. The directed sorting synthesis technique combines the advantages of parallel synthesis systems and split-and-pool synthesis to generate compound libraries in short times. The non-invasive Rf tag labelling technique allows the microreactors to be sorted between individual reaction steps in a chemical synthesis. The use of the Rf tags also provides a convenient and positive identification of compounds for archival and storage purposes at the conclusion of the synthesis procedure.

Each of these building blocks will present a structural diversity, such as the final library will contain around 30,000 compounds. Their synthesis (FIG. 9) is designed such as to be modular, and therefore amenable to solid-phase automated synthesis and screening. As illustrated by FIG. 9 which is not intended to be limiting, a candidate compound comprising a peptide-like, melatonin-like and nicotine-like domain may be synthesized starting with a short peptide-like backbone. The peptide backbone preferably contains 3-6 amino-acid-like molecules and more preferably three amino-acid-like molecules. A melatonin-like domain is chemically conjugated to the N-terminal end of the peptide-like domain. A nicotine-like domain is then added as a side chain to one of the amino-acid-like elements in the peptide-like domain. Alternatively, the nicotine-like domain is added to the amino functionality of the melatonin-like domain to generate the amide.

Compounds made from only two of the above-described chemical domains can also be made using this method. To synthesize a compound comprising a peptide-like and melatonin-like domain, the melatonin-like domain is added to the N-terminal end of the peptide-like domain or alternatively to the c-terminal end. To synthesize a compound comprising a peptide-like and nicotinic-like domain the nicotine-like domain is added as a side chain to one of the amino-acid-like elements of the peptide-like domain. To synthesize a compound comprising a melatonin-like and nicotine-like domain, the nicotine-like domain is chemically conjugated to the amino functionality of the melatonin-like domain to generate the amide. Once the building blocks are obtained and the solid phase synthesis validated, the throughput can reach approximately 30,000 compounds per month.

B. Methods of Screening

Different types of screening systems can be used to assess the activity of candidate compounds. The classical Thioflavin assay, in which Aβ fibers are pre-formed and their dissolution in presence of synthesized compounds is followed by the change of fluorescence of Thioflavin. This can be achieved in 384 well plates. This method has recently been used to screen molecules for Aβ dissolution. (Blanchard et al., Proc Natl Acad Sci USA. Oct. 5, 2004;101(40):14326-32). Another in vitro assay is used for screening where direct detection of Aβ complexes of library members with mass spectrometry is employed. (Wang et al., 1996 J Biol Chem, 271(50): 31894-31902). Using a mass spectrometric approach, a series of candidate compounds can be examined for binding to Aβ. The ligands are evaluated for their ability to bind to and stabilize the tetrameric structure, their cooperativity in binding. Using this method, Aβ and its variants are immuno-isolated with Aβ-specific monoclonal antibodies. The identities of the Aβ variants are determined by measuring their molecular masses using matrix-assisted laser desorption ionization time-of-flight mass spectrometry. The levels of Aβ variants are determined by their relative peak intensities in mass spectrometric measurements by comparison with internal standards of known identities and concentrations. This method is used to examine the Aβ species in conditioned media in vitro. In addition to human Aβ-(1-40) and Aβ-(1-42), more than 40 different human Aβ variants may be identified. This method allows direct measurement of each individual peptide in a peptide mixture and provides comprehensive information on the identity and concentration of Aβ and Aβ variants. Hardy J. 1992, Nat. Genet. 1:233-234; Swiss-Prot P05067, pp 13-17).

A third in vitro assay is a well-less, microarray-based assay. In this assay, a mixture containing a small fraction of fluorescently labeled Aβ and non-labeled Aβ is incubated with a glass slide that is patterned with a hydrophobic coating. The Aβ fibers incorporating the fluorescent peptide will be deposited at each location on the microarray. The proportion of the fluorescent peptide will be equal at each location (address). Then, each address on the well-less microarray will be incubated with a solution containing the compound to be screened. After a given and carefully chosen time, the microarray will be washed and the remaining fluorescence will be measured at each address. The throughput of this screening method can be up to 100,000 compounds per day per working station.

An in vivo assay has also been recently published (Wigley, W. C. et al., Nature Biotechnol. 2001, 19:131-136). This general method assesses the solubility and folding of proteins in vivo. The basis of this in vivo assay is structural complementation between the alpha- and omega-fragments of beta-galactosidase (beta-gal). Fusions of the alpha-fragment to the C terminus of target proteins with widely varying in vivo folding yield and/or solubility levels, including the Alzheimer's Aβ peptide and a non-amyloidogenic mutant thereof, reveal an unambiguous correlation between beta-gal activity and the solubility/folding of the target. Thus, protein solubility/misfolding can be monitored in vivo by structural complementation, and is used to screen for compounds that influence the solubility of Aβ peptide.

This invention is further illustrated by the following examples, which are not to be construed in any way as imposing limitations upon the scope thereof.

### EXAMPLE I

### Synthesis of Small Molecule Compounds for Screening for Activity on Amyloid Beta Dissolution

Mesh micro-reactors are loaded with resin for solid phase synthesis of the compound. Synthesis takes place by allowing the reagents to flow through the mesh of the micro-reactor. Virtually any loose resin chemistry can be performed in the micro-reactor. Normal glassware is used for heating, refluxing, cooling etc. of groups of micro-reactors that have been selected by their Rf tags. About 1 ml of reagent per micro-reactor is required for the reaction. When the diversity reaction is complete, the micro-reactors are pooled for a common wash step. The micro-reactors are then sorted using their Rf tags to a reaction vessel for the next step containing the next building block reagent for the second diversity reaction. After all diversity steps are complete, the AccuCleave-96 cleavage station cleaves the compound from the solid support and collect it in a variety of formats for final archive. The Synthesis Manager software records the compounds structure and final archive location for easy retrieval.

### EXAMPLE 2

### Screening of Small Molecule Compounds for Activity on Amyloid Beta Dissolution

To measure β-sheet formation, Thioflavin T (ThT) is added to samples and fluorescent measurements are read as follows. To measure calcium, the ratiometric calcium dye fura-2 is loaded into a mouse neuronal cell line, CATH.a (CRL-11179; American Type Culture Collection) and plated on acid-washed poly-D-lysine-coated glass coverslips. Cytosolic calcium concentrations are measured in Tyrode's solution/2 mM calcium. Rapidly alternating measurements of fura-2 (excited at 340 and 380 nm) are performed by measuring emission at 516 nm using the special Fura2-Fluo3 filter set 7400 (Chroma Technology, Brattleboro, Vt.), an Axiovert 100 inverted microscope (Zeiss), and a photometry system (DeltaRam model, Photon Technology International, Lawrenceville, N.J.). Each sample is set up in a 1.5-ml Eppendorf tube and incubated in a 37° C. water bath. If stirring of the sample during incubation is required, it is set up in a 2.0-ml screw-cap vial (Corning) with a magnetic stir bar and placed on a stirrer in the 37° C. room.

In “coincubated” samples, the Aβ-42 is added first, then the candidate small molecule is added, and the sample is incubated for the designated amount of time (usually 48 h). If it is an “added after aggregation” sample, the candidate small molecule is added after the Aβ-42 is incubated. The ThT dye is always added last and after incubation. For measurement, each sample is split into four wells (100 μl per well) of a 96-well black-bottom plate (catalog no. 35-3943, VWR International, Westchester, Pa.). ThT fluorescence is measured at room temperature in a Fluoroskan II at Eₘ=444 nm and Eₓ=510 nm or a FLUOstar Optima plate-reader (BMG Lab Technologies, Durham, N.C.) at Eₓ=440 nm and Eₘ=480 nm. The ThT fluorescence spectrum is measured in an f4500 spectrofluorimeter (Hitachi, Tokyo) at Eₓ=435 nm and Eₘ=450-550 nm.

### EXAMPLE 3

### Administration of Small Molecule Compounds in a Mouse Model to Assess Effects on Disease Progression and Aβ Dissolution

APP transgenic Tg2576 mice (Taconic Farms, Germantown, N.Y.) at 11 months old are fed the drug-supplemented chow ad libitum for 16 weeks. There are five animals in each treatment group. Animals are housed singly in individual cages, and their body weight and food consumption are monitored weekly. There are no significant differences in the amount of chow consumed or in weight of the mice during the experimental period, either within or between treatment groups. The food consumption of animals in this experiment is 5 gm of rodent chow per day per animal, resulting in a final dose of candidate small molecule compound of 20 mg·kg⁻¹·d⁻¹. The dosage of the candidate small molecule can be titrated at various doses for clearance and toxicity studies.

Behavioral Testing.

Spatial reference learning and memory is tested using the conventional Morris water maze (Morris, 1984, J Neurosci Methods. May 11(1):47-60) in a group of Tg2576 mice and controls across their 2 year life span. Mice are grouped into four age ranges corresponding to four stages in Tg2576 plaque pathology and Aᵢₙₛₒₗ levels: (1) very young mice, 4-5 months, before the appearance of Aᵢₙₛₒₗ or plaques; (2) young mice, 6-11 months, during the initial appearance of Aᵢₙₛₒₗ and both amyloid plaques and punctate A deposits; (3) middle-aged mice, 12-18 months, during the extensive deposition of plaques when Aᵢₙₛₒₗ levels are rising rapidly; and (4) old mice, 20-25 months, at a time when A is leveling off and amyloid loads are comparable to those in Alzheimer's disease. Punctate A deposits much smaller and sparser than mature plaques appear at 6-8 months, whereas amyloid plaques appear at 9-12 months and increase to numbers similar to those seen in Alzheimer's patients by 16-20 months (Irizarry et al., J Neurosci. Sep. 15, 1997; 15;17(18):7053-9; Kawarabayashi et al., J Neurosci. Jan. 15, 2001;21(2):372-81). Approximately equal numbers of male and female mice are tested. All mice are naive and tested in a coded manner.

The water maze is tailored to Tg2576 mice in a manner that enables the detection and distinguishing of all stages of memory loss. This protocol provides the sensitivity, specificity, and dynamic range needed to measure changes that are subtle early in life and gross late in life. Interpolation of probes during training provided sensitivity. Adoption of exclusion criteria for performance deficits gives specificity. Training extensively lends dynamic range. The assignment of mean probe scores (MPSs), which is the mean percentage time spent by a mouse in the target quadrant during the three probe trials, improves quantification of cognitive performance of individual mice for correlations with molecular markers and provides a single measure with a broad dynamic range. Protocols for Tg2576 mice in other strain backgrounds may need to be adjusted for strain-specific differences in rates of learning and performance deficits.

The water maze is a circular 1 or 1.2 m pool filled with water at 25-27° C. and made opaque by the addition of nontoxic white paint. The pool is placed amid fixed spatial cues consisting of boldly patterned curtains and shelves containing distinct objects. Mice are placed in a beaker and gently lowered into the water facing the wall of the pool. Mice first undergo visible platform training for 3 consecutive days (eight trials per day), swimming to a raised platform (a square surface 12×12 cm²) marked with a black and white striped pole. Visible platform days are split into two training blocks of four trials for statistical analysis. During visible platform training, both the platform location (NE, SE, SW, or NW) and start position (N, NE, E, SE, S, SW, W, or NW, excluding the positions immediately adjacent to the platform) are varied pseudorandomly in each trial.

Pseudorandomization ensures that all positions are sampled before a given position is repeated. Hidden-platform training is conducted over 9 consecutive days (four trials per day), wherein mice are allowed to search for a platform submerged 1.5 cm beneath the surface of the water. Mice failing to reach the platform within 60 sec are led to the platform with a metal escape scoop. During hidden-platform trials, the location of the platform remains constant (NE, SE, SW, or NW), and mice enter the pool in one of the seven pseudorandomly selected locations (N, NE, E, SE, S, SW, W, or NW, excluding the position immediately adjacent to the platform). After each hidden platform trial, mice remain on the platform for 30 sec and are removed from the platform and returned to their home cage with the escape scoop. Mice quickly learn to associate the scoop with escaping from the pool and consistently orient to or follow the scoop on its appearance. The ability of mice to orient to or follow the escape scoop represents independent measures of vision and attention. At the beginning of the 4th, 7th, and 10th day of hidden platform training, a probe trial is conducted in which the platform is removed from the pool and mice are allowed to search for the platform for 60 sec. All trials are monitored by a camera mounted directly above the pool and are recorded and analyzed using a computerized tracking system (HVS image, Hampton UK). Further analysis is done using Wintrack. (D. Wolfer, Zurich, CH).

The MPS is calculated for each mouse and used to assess retention of spatial information in the Morris water maze. By integrating information from the intercalated probes, the MPS represents a measurement of learning similar in concept to the previously described learning index (Gallagher et al. Behav Neurosci. August 1993;107(4):618-26.), which samples memory at different stages of learning. Similar statistical results are found with MPS, the learning index and learning score (the weighted sum of percentage time spent in the target quadrant during probe trials).

Histological Analysis

At the end of the experimental period, animals are euthanized and the brain is dissected and the hemispheres separated along the midline. Half of the brain is frozen on dry ice for Aβ ELISA analysis. The other hemisphere was frozen in OCT medium for histological study. Coronal sections (14 μM) are cut on a cryostat microtome. Sections are thaw mounted onto Fisher “plus” microscope slides, air-dried, and then stored at −20° C. until use. Sections are warmed to room temperature and fixed in 4% paraformaldehyde/0.1 M phosphate buffer, pH 7.2, for 1 hr. The endogenous tissue peroxidase activity is quenched by incubation with 3% H₂O₂ in PBS for 20 min. For Aβ immunohistochemistry, sections are subsequently incubated with 88% formic acid for 20 min to expose the Aβ epitope. Sections are then incubated with blocking solution (3% normal goat serum, 5% normal horse serum, 0.25% carrageenan lambda, 0.1% Triton X-100 in PBS) for 1 hr. The primary antibodies used are biotinylated mouse anti-human Aβ monoclonal antibody 4G8 (Signet Pathology System, Dedham, Mass.) at 0.5 μg/ml, in the blocking solution overnight at 4° C. The antigen is detected by secondary antibody where needed and Aβ C-DAβ method. Sections are dehydrated and coverslipped with mounting medium.

### EXAMPLE 4

### Administration of Small Molecule Compounds to Humans Expressing Symptoms of Alzheimer's Disease

Participants are selected with probable Alzheimer's disease according to National Institute of Neurological and Communicative Disorders and Stroke and the Alzheimer's Disease and Related Disorders Association criteria. Patients with moderate to severe Alzheimer's disease are randomized to receive either the test compound or placebo. The effective dosage amount can be titrated to higher or lower amounts based on data obtained from animal models. Such methods to extrapolate animal dosages to humans are known in the art. (Mahmood., Am J Ther 2001 8(2):109-116; Wajima et al. J Pharm Sci 2003 92(12):2427-2440). Baseline demographics are normalized between the drug and placebo groups. Dosage regimens are structured to assess the effects of a low dose or a high dose on symptoms of Alzheimers disease. The drug is administered to patients for a one year period. Patients are monitored for changes in behavior and memory during the course of the study at 0, 1, 3, 6, 9, and 12 months. Cognitive tests are used to assess the patient's performance in cognitive tasks. Such tests include the Alzheimer's Disease Assessment Scale (ADAS-Cog; Rosen et al., 1984 Am J Psychiatry 14:1356-64), Mini-Mental State Exam (MMSE; Folstein et al., 1975, J. Psychiatry Res 12:189-98), Clinical Dementia Rating (CDR; Hughes et al., 1982, Br. J Psychiatry 140:566-72), and Wechsler visual memory test (WECHSLER, D. (1981, 1987, 1989, 1991). Manuals for the Wechsler Adult Intelligence Scale—Revised). The presence of biochemical markers is determined by assay of cerebrospinal fluid withdrawn through lumbar puncture. Such biochemical markers include tau protein and amyloid beta peptide 42. Brain volume is also measured with magnetic resonance imaging.

It is to be clearly understood that resort may be had to various other embodiments, modifications, and equivalents thereof which, after reading the description herein, may suggest themselves to those skilled in the art without departing from the spirit of the present invention and/or the scope of the appended claims.

We claim:

1. A method for designing small molecule compounds for solubilizing amyloid protein comprising:
creating a combinatorial library of compounds possessing at least two chemical domains selected from a peptide-like domain, a melatonin-like domain and a nicotine-like domain, and
screening the combinatorial library for compounds that bind to and solubilize amyloid protein.

2. The method of claim 1 wherein the compound comprises a peptide-like domain, a melatonin-like domain and a nicotine-like domain.

3. The method of claim 1 wherein the compounds bind to the helix-loop-helix structure of amyloid protein.

4. The method of claim 3 wherein the compounds bind non-covalently to amyloid protein.

5. The method of claim 1 wherein the compounds in the combinatorial library are created by solid phase synthesis.

6. The method of claim 1 wherein the compounds have a binding energy with amyloid protein of between 1 and 20 kcal/mol.

7. The method of claim 6 wherein the binding energy is between 5 and 15 kcal/mol.

8. The method of claim 7 wherein the binding energy is between 7 and 12 kcal/mol.

9. The method of claim 8 wherein the binding energy is 8 kcal/mol.

10. The method of claim 1 wherein the compound comprises a melatonin-like domain and a peptide-like domain wherein
the melatonin-like domain is covalently bonded to the N-terminus or C-terminus of the peptide-like domain.

11. The method of claim 10 further comprising covalently bonding a nicotine-like domain to the peptide-like domain or the melatonin-like domain.

12. The method of claim 11 wherein the melatonin-like domain is covalently bonded to the N-terminus of the peptide-like domain and
the nicotine-like domain is covalently bonded to the melatonin-like domain.

13. The method of claim 11 wherein the melatonin-like domain is covalently bonded to the N-terminus of the peptide-like domain and
the nicotine-like domain is covalently bonded to the peptide-like domain.

14. The method of claim 1 wherein the compound comprises a nicotine-like domain and a melatonin-like domain or a peptide-like domain wherein
the nicotine-like domain is covalently bonded to either the melatonin-like domain or the peptide-like domain.

15. The method of claim 14 wherein the nicotine-like domain is covalently bonded to a melatonin-like domain.

16. The method of claim 14 wherein the nicotine-like domain is covalently bonded to a peptide-like domain.

17. The method of any of claims 1-16 wherein the compounds are useful for treating symptoms of Alzheimer's disease.

18. A composition made by the process of designing small molecule compounds for solubilizing amyloid protein comprising:
creating a combinatorial library of compounds possessing at least two chemical domains selected from a peptide-like domain, a melatonin-like domain and a nicotine-like domain, and
screening the combinatorial library for compounds that bind to and solubilize amyloid protein.

19. The composition of claim 18 wherein the compound comprises a nicotine-like domain, a melatonin-like domain and a peptide-like domain.

20. The composition of claim 18 wherein the compound comprises two chemical domains wherein the chemical domain is a nicotine-like domain, a melatonin-like domain or a peptide-like domain
